(19) United States
(12) Patent Application Publication
(10) Pub. No.: US 2024/0181272 A1
(43) Pub. Date: Jun. 6, 2024

(54) Antimicrobial Dose Delivery System and Method

(71) Applicant: Robert John Ireland, Forest Grove, OR (US)

(72) Inventor: Robert John Ireland, Forest Grove, OR (US)

(21) Appl. No.: 18/073,385

(22) Filed: Dec. 1, 2022

(51) Int. Cl. A61N 5/06

(52) U.S. Cl. CPC A61N 5/0624; A61B 2017/00725

(57) ABSTRACT

The present invention relates to a simple-to-use, phototherapy dose delivery system. More particularly, a two-component irradiator having a first component being antimicrobial light source and the second being an expendable, hollow, beam sleeve. The beam sleeve being constructed to slide partially over a bezel end of the light source thereby capturing and transforming the source light into a contained, homogeneous treatment spot of calibrated treatment intensity. The calibrated intensity enables protocols and dose times to become universally the same for all irradiators once paired with a fitting beam sleeve, regardless of spot size or spectra. In practice the beam sleeve defines the treatment spot diameter, gauges the working distance, sets the dose rate, all while blocking stray light from disrupting vision. In this modular manner the light source and beam sleeve work similarly as a syringe and needle; when used together the predetermined dose and rate are administered to a precise location in accurate amounts.

### BACKGROUND OF THE INVENTION

### 1) Field

Disclosure relates to the prophylactic and therapeutic application of light to cause antimicrobial effects in or in the body. More particularly, the present invention relates to a simplified photonic dose system having reusable and expendable beam forming bodies that slide over standardized light emitting diode (LED) sources to form calibrated irradiator units that produce standardized dosage rates enabling universal dose times regardless of spectra, size, or the source intensity.

### 2) Description of Related Art

Antibacterial phototherapy [AP], antimicrobial photodynamic therapy [aPDT], and photobiomodulation therapy [PBMT] are all treatment methods primarily related to this disclosure, and more particularly to the irradiators that are operable to deliver an accurate dose of calibrated light in or on the body to cause a beneficial effect. Low-intensity LEDs deliver bactericidal dose over a long period of time and are considered safe for use on or within the body without risking cell harm regardless of the exposure duration. However, the very long exposure times needed to deliver a bactericidal dose prevents adoption into wound care. Recently high-intensity LEDs configured discretely in a reflector, array, or in chip on board (COB) module overcome the historic slow dose rate problem, however new challenges arise with high intensity such as shrouding stray light, ensuring patient comfort, and avoiding mammalian cell damage caused by overdose.

For disclosure clarity, terms of art used herein are addressed as follows.
Wavelength is discussed in nanometers (nm) and defines the spectra produced by the irradiator which often includes a band of wavelengths being produced. The spectra primarily discussed herein is 365 nm-470 nm as being both bactericidal and considered skin-safe, with 405 nm being at the peak susceptibilities of pathogens like Staph. and M.R.S.A.
Intensity (also referred to in the arts as fluence, irradiance, and energy density) is measured in various ways but the scientific paper and this disclosure will be disclosed in milliwatts per centimeter squared (mW/cm2) representing the intensity output of the irradiator regardless of wavelengths produced. Intensities exceeding 100 mW/cm2 can cause discomfort when shined into a wound, comfort threshold as discussed herein means under 100 mW/cm2.
Photonic dose is calculated by multiplying treatment spot intensity with dose time, dose referred herein as joules per centimeter squared (J/cm2). The time of exposure required to deliver a particular dose is a function of dose in Joules divided by intensity measured in mW/cm2. A dose of 36 J/cm2 of 405 nm light has been found safe, however 54 J/cm2 has been found to cause cell death.
Working distance is the distance in centimeters (cm) between the light source and the patient from which intensity is measured to calculate dose time. The working distance can be increased or decreased to adjust intensity and dose time.

Visible spectra violet to blue LEDs (400 nm-470 nm) have been scientifically studied and medically used to produce antimicrobial effect in wounds. In the December 2012 medical publication “Antimicrobial Agents and Chemotherapy” a 415 nm experiment at 14.6 mW/cm2 intensity completely cured burned and terminally infected lab mice. However, to deliver the target 54 J/cm2 dose the exposure time needed exceeded an hour:
Blue light rescued mice from otherwise lethal P. aeruginosa burn infection. FIG. 5A and B show the successive bioluminescence images of representative full-thickness mouse burns (1.2 cm by 1.2 cm) infected with 3 106 CFU of luminescent P. aeruginosa, with and without blue light therapy, respectively. Blue light (415 nm) was delivered at 30 min after bacterial inoculation. Bacterial luminescence was completely eliminated after 55.8 J/cm 2 blue light had been delivered (62 min of illumination at an irradiance of 14.6 mW/cm 2) (FIG. 5A), while in the untreated mouse burn, infection steadily developed with time (FIG. 5B), and the mouse died at 72 h (day 3) after bacterial inoculation.

Luminescent P. aeruginosa was detected in the blood culture of the dead mouse, indicating that the mouse died of sepsis The rescued mice study examples LEDs projecting uncontained diverging beams used to irradiate the entire mouse body and surrounding areas. This method worked but was highly inefficient as the stray light not directed to the wound was wasted and polluted the treatment space, which leads to problems when the intensity is increased. Had the researchers been able to direct all light produced by the LEDs directly into the mouse wounds, the time to dose could have been reduced. If intensity could be increased by 10×, the experimental dose time of 62 minutes could have been reduced to 6.2 minutes.

A prior art patent example of an antimicrobial irradiator using a tubular structure to help administer dose is taught by Eltorai in US20190168023A1 “a unique solution to the problem of catheter-associated infections by providing a catheter with an optically transparent wall and a light source configured to emit any antimicrobial visible light, such as visible spectrum violet-blue 405 nm or 415 nm light, through the optically transparent wall. Because of the antimicrobial properties of violet-blue light, the risk of bacterial infection through the use of the catheter is reduced.” Eltorai teaches low intensity treatment being administered through the catheter tube to adjacent tissues inside the body but does not anticipate high intensity light delivered from a working distance outside the body. Gill et. al. in U.S. Pat. No. 7,182,597B2 from the UV curing arts teaches a bent tubular structure that acts as an off-axis light guide for redirecting UV light from a source outside the body into a light-curable compound located in the mouth of the patient. However useful in guiding light Gill's tubular structure does not calibrate intensity of other sources or provide efficient energy transfer from the source to the target. Nothing in the last one-hundred years of antimicrobial phototherapy has there been a standardizing solution to delivering an accurate dose of light.

What has happened is that LED technology leaped ahead in cell-safe antimicrobial spectra outputs before the medical community had time to develop accurate and standardized dosing methods. What is needed is a “Syringe and Needle” approach like was provided by Blaise Pascal in 1650. Pascal solved the medical community problem of delivering liquid drugs into a human vein by providing a modular system of standardized components. The needle gauged the size of injection site and calibrated the rate of drug flow with the needle's internal diameter. The syringe contained and delivered the measured dose through the needle into the vein. This type of simplified modular approach is needed in the phototherapy arts enabling practitioners to deliver photonic treatment accurately, precisely, and in a standardized way.

### SUMMARY OF INVENTION

A photonic dosing system and method that involves two-components: an antimicrobial light source and a corresponding calibrating beam sleeve. Before treating, the beam sleeve (referred herein as a body) is partially slid over the bezel end of the source to fill and seal a first portion of the body, leaving remainder second portion of the body empty and protruding in the direction of the source light. The second portion's interior acting as a beam forming element that reflects, captures, and directly passes the source beam light to form a contained homogenized treatment spot of calibrated intensity. The calibrated intensity enables simplified operation and universally consistent treatment protocols between selectable light sources. In this way the practitioner selects the light source having the desired wavelength and spot size then engages the calibrating body that slides over the bezel diameter to govern intensity to a comfortable level and set dose time to universally used protocols.

The objects of the present invention include, but are not limited to:
To provide a modular irradiator system of standardized components to enable universal dosing protocols
To simplify training, maintenance, and operation
To protect vision during in-clinic and prescriptive home phototherapy
To provide the most effective and least costly irradiator solution

The advantages of the present invention include, but are not limited to:
Having ability to increase intensity to reduce dose times
Having a physical working distance gauge to provide consisted dosing
Having a calibrated diameter to provide universal beam sleeve use between components
Having calibrated intensity which standardizes efforts to develop treatment protocols

Plethora of objects, features, aspects, and other advantages of the exemplary embodiments of the present invention will become more apparent from the following detailed description, along with the accompanying drawings in which like numerals represent like components.

### BRIEF DESCRIPTION OF THE DRAWINGS

FIG. 1 is a perspective view of the irradiator unit prepared for delivering a calibrated dose according to one embodiment of the disclosure.

FIGS. 2 and 3 are in-use perspective views of the embodiment shown in FIG. 1.

### DETAILED DESCRIPTION OF THE INVENTION

The following detailed description is of the best contemplated embodiment and simplest mode of carrying out the invention with least number of components. This description and illustration of embodiments are not to be taken in a limiting sense but is made merely for the purpose of illustrating the general principles of the invention, how it is used, and the problems of prior art irradiators solved.

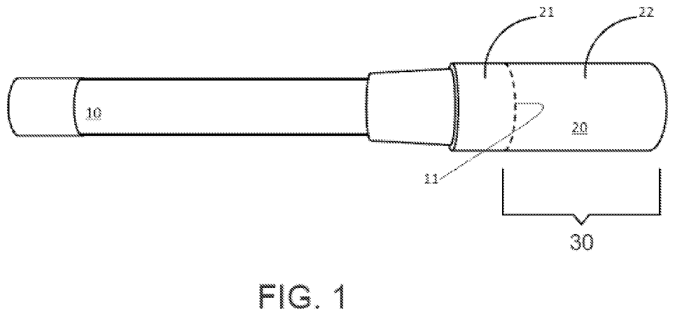

As depicted, in FIGS. 1-3 a preferred embodiment of a modular irradiator for delivering a calibrated light dose is illustrated. FIG. 1 depicts a phototherapy light source 10 having a cylindrical bezel end 11 that slidingly engages within a beam forming hollow cylindrical body 20 to fill and seal within a first portion 21 leaving a remaining second portion 22 empty and protruding in the direction of the light source 10. The light source 10 having one or more LEDs producing a standardized output that correlates to the diameter of the bezel end 11. The sealing engagement between the first portion 21 interior and said bezel end 11 exteriors are such that stray light is blocked and the body 20 remains attached during treatment. Like a needle taken off a syringe post injection the body 20 also easily removes with one hand to be replaced before treating next patient.

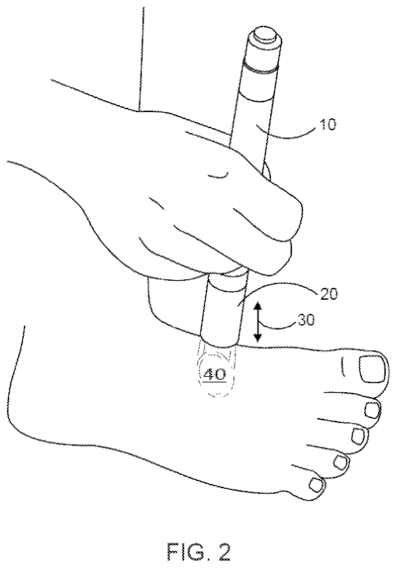

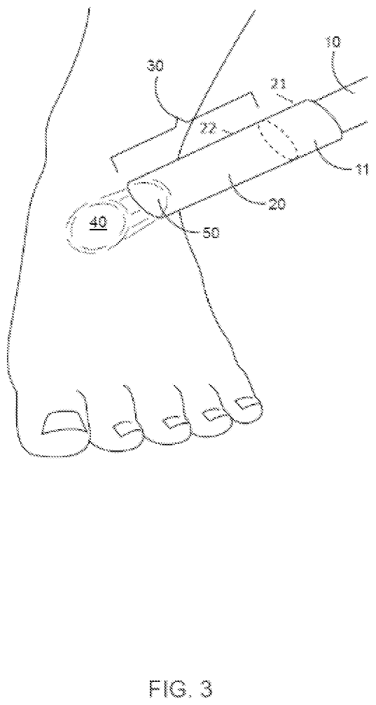

Most of the light produced by the source 10 transfers unobstructed through the body 20 interior with remainder being conditioned by the second portion 22. The interior of said second portion 22 having reflective and diffusing properties that transform the raw antimicrobial light projecting from said source 10 into a contained treatment spot 40 (shown in FIGS. 2-3) of calibrated intensity. Calibrated meaning that the intensity of said treatment spot 40 is set by the working distance 30 gauged by the length of the second portion 22 which corresponds to the diameter of the first portion 21. In this way the second portion 22 length provides a physical gauge to prevent the intensity from exceeding comfortable levels during treatment.

Preferably constructed of medical grade silicone to have durable, flexible, stretchable characteristics, said body 20 can also be constructed of any resilient material capable of holding a hollow shape and capable to sealingly form over said bezel end 11 to block stray light and secure said body 20. The shape of the body 20 is shown hollow straight and cylindrical and installed on-axis in the preferred embodiment to maximize intensity. However, the diameter of the first portion 21 and diameter of the second portion 22 may be of different in other embodiments.

In the preferred embodiment at least the second portion 22 is constructed of photoluminescent materials that absorb light in the visible or ultraviolet wavelengths and then re-emit in visible wavelengths. This effect provides visible notice and warning when treating with invisible UV light or hard to see Violet 405 nm to prevent accidental exposure. After treatment the residual glow acts as a visual que “contaminated” and to replace body 20 with a clean uncontaminated one. The secondary glow also provide secondary irradiation at different wavelengths than the source. 10.

As illustrated in FIG. 2 said second portion 22 transforms the beam produced from said source 10 into a contained treatment spot 40 having calibrated intensity. The protruding second portion 22 provides visual and physical gauge for maintaining said working distance 30 during treatment, proves especially helpful when dosing a non-compliant, fidgety patient. The protruding second portion 22 is easily shortened if the intensity is too low or lengthened if less intensity is desired. Those skilled in the phototherapy arts understand that the working distance 30 sets the intensity for a diverging source 10. Move it farther away and intensity decreases, and vice versa. Maintaining working distance 30 during the treatment session improves the accuracy of dose by keeping the intensity consistent. Having the working distance 30 physically gauged helps prevent accidental contact or excessive intensity caused by getting the bezel end 11 too close to the patient.

FIG. 3 illustrates the embodiment having the hollow elongate body 20 partially filled with a translucent medium 50 that is hydraulically held within the second portion 22 like an optical lens. In this way said source 10 light can be focused, attenuated, filtered, and diffused by said medium 50 enabling adaption of very intense sources 10 for use on sensitive areas. Photosensitizers and photoactivated compounds such as methylene blue may be added to the medium 50 to be dispensed while dosing to act as a multiplier. In practice said second portion 22 is loaded with said medium 50 having photosensitizer compounds, then said second portion 22 is pressured by squeezing from the sides to dispense said medium 50 into area being irradiated by said treatment spot 40. One can envision sources 10 of varying wavelength and diameter all cooperatively working with calibrating bodies 20 that come pre-loaded with either or both optical forming gel and therapeutic compounds that, when energized by the source 10, have synergistic healing effects. A photonic pharmacy of wavelength selectable sources 10 that, when paired with a calibrated bodies 20, produce standardize dose rates enabling universally used treatment times.

It would be appreciated by those skilled in the art that various changes and modifications can be made to the illustrated embodiments without departing from the spirit of the present invention. All such modifications and changes are intended to be within the scope of the present invention, except as limited by the scope of the claims.

What is claimed is:

1. A phototherapy system for delivering a calibrated dose of light, comprising:
a light source configured to project a source beam from a cylindrical bezel end;
a beam forming cylindrical body constructed with open ends having a first portion and a second portion;
said first portion arranged to slidingly engage over said bezel end to sealingly fill said first portion while leaving said second portion empty; and
said second portion aligned to pass a majority of said source beam such that a contained treatment spot of calibrated intensity projects from said open end of said second portion.

2. The apparatus of claim 1, wherein said body is constructed of light blocking materials to prevent stray light from escaping into the treatment space.

3. The apparatus of claim 1, wherein said second portion is constructed of materials that reflect, absorb, and diffuse said source beam resulting in a more homogenized treatment spot.

4. The apparatus of claim 1, wherein at least said second portion is constructed of photoluminescent materials that absorb light in the visible or ultraviolet wavelengths and then re-emit in visible wavelengths.

5. The apparatus of claim 1, wherein said body is constructed and arranged such that said body's interior is reflective to spectra between 365 nm to 470 nm thereby increasing intensity of said treatment spot over said source beam intensity.

6. The apparatus of claim 1, wherein said second portion hydraulically holds a medium that diffuses and filters said source beam thereby attenuating intensity of said treatment spot.

7. The apparatus of claim 1, wherein said bezel end further comprises a diameter equating to a standardized source beam output operable for universal dose time calibration when used with said body.

8. The apparatus of claim 1, wherein said body further comprises an interior diameter equating to a standardized second portion length operable for universal gauging of working distance for uniform dose times.

9. The apparatus of claim 1, wherein said second portion length is arranged to physically limit intensity of said treatment spot from exceeding comfortable levels.

10. A phototherapy method for delivering a calibrated dose of contained light, the method comprising:
engaging a removable, hollow, cylindrical body partially over a bezel end of a phototherapy light source leaving the remainder protruding to transform the source beam into a contained treatment spot of calibrated intensity that is gauged a working distance away.

11. The method of claim 10, wherein the hollow elongate body blocks stray light to protect vision.

12. The method of claim 10, wherein said body having internal walls that pass, reflect, and diffuse said source beam resulting in a homogenized treatment spot intensity.

13. The method of claim 10, wherein said body is constructed of photoluminescent materials that absorb light in the visible or ultraviolet wavelengths and then re-emit in visible wavelengths.

14. The method of claim 10, wherein said second portion is constructed from materials that reflect light in the spectra between 365 nm to 470 nm thereby increasing said treatment spot intensity over said light source.

15. The method of claim 10, wherein said second portion hydraulically holds a medium that diffuses and filters light thereby attenuating intensity of said treatment spot.

16. The method of claim 10, wherein said bezel end further comprises a diameter equating to a standardized diameter relative to source beam output operable using universal dose times.

17. The method of claim 10, wherein said body further comprises an interior diameter equating to a standardized second portion length operable for gauging working distance.

18. The method of claim 10, wherein said second portion length is arranged to gauge working distance thereby physically limiting intensity of said treatment spot from exceeding comfortable levels.
